(19) United States
(12) Patent Application Publication
(10) Pub. No.: US 2018/0169287 A1
(43) Pub. Date: Jun. 21, 2018

(54) AIR-CONDITIONING DEVICE

(71) Applicants: Relyon Plasma GmbH, Regensburg (DE); EPCOS AG, Munich (DE)

(72) Inventors: Stefan Nettesheim, Regensburg (DE); Dominik Burger, Alteglofsheim (DE); Georg Kuegerl, Eibiswald (AT); Markus Puff, Graz (AT)

(21) Appl. No.: 15/894,153

(22) Filed: Feb. 12, 2018

Related U.S. Application Data
(63) Continuation of application No. PCT/IB2016/054843, filed on Aug. 11, 2016.

(30) Foreign Application Priority Data
Aug. 12, 2015 (DE) 102015113276.1

(51) Int. Cl. A61L 9/12; A61L 9/22; A61L 9/04; B01D 53/66

(52) U.S. Cl. CPC A61L 9/122; A61L 9/22; A61L 9/046; H05H 1/2475; A61L 2209/132; A61L 2209/212; B01D 53/66

(57) ABSTRACT

The invention relates to an air-conditioning device for a space to be air-conditioned. The air-conditioning device has a housing including an air supply and an air discharge. In the housing, a flow path is formed between the air supply and the air discharge. In the flow path, at least one heat exchanger, a blower, and a means for generating an excited gas are provided. Furthermore, a surface coating is provided in the flow path, which is configured for the catalytic decomposition of the ozone content of the excited gas, and which is arranged downstream of the means for generating the excited gas.

### CROSS-REFERENCE TO RELATED APPLICATIONS

The present application is filed under 35 U.S.C. §§ 111(a) and 365(c) as a continuation of International Patent Application No. PCT/IB2016/054843, filed on Aug. 11, 2016, which application claims priority from German Patent Application No. DE 10 2015 113 276.1, filed on Aug. 12, 2015, which applications are incorporated herein by reference in their entireties.

### FIELD

The present application relates to an air-conditioning device, in particular an air-conditioning device for use in a motor vehicle. The air-conditioning device is for a space to be air conditioned and comprises a housing with an air supply and an air discharge. Between the air supply and the air discharge, a flow path is formed, wherein at least one heat exchanger and a blower are provided in the flow path.

### BACKGROUND

The German utility model DE 20 2012 010 239 U1 discloses a device for air purification. The device comprises a housing, an electrostatic charging section, and an odor filter. An airflow is directed in a housing from the air inlet to a plasma filter comprising the electrostatic charging section and the odor filter to the air outlet. The charging section comprises at least one plate-shaped plasma electrode for generating the plasma.

The German patent application DE 10 2014 107 805 A1 relates to an arrangement for filtering air, wherein in an air duct, a plasma cell, a catalyst, and an impeller are also provided.

The German patent application DE 10 2004 034 432 A1 relates to a filter and/or a filter arrangement for a motor vehicle air-conditioning system and/or a motor vehicle ventilation system. The filter is traversed by a flow of air driven, in particular, by a blower, and the filter converts one or more pollutants into harmless substances. The filter consists at least partially of a protein-rich material which is coated with a catalytic agent.

The German Patent Application DE 11 2013 001 665 T5 relates to a device for cleaning the air-conditioning system of vehicles. For this purpose, a catalytic radiation ionization is used. By means of a UVX bulb surrounded by a noble metal alloy, air or oxygen is converted into a purifying plasma containing hydroxyl radicals and hydrogen peroxide.

The translation DE 60 304 432 T2 of European patent EP 1 435 306 discloses an air air-conditioning device for a vehicle. A module of the air-conditioning device allows the filtering of gaseous compositions, e.g., volatile organic compounds and microorganisms that are moved through the air of the air-conditioning device. For this purpose, a plasma-catalytic detoxification module is provided, which is combined with an electrostatic detoxification module.

### SUMMARY

An object of the invention is to provide an air-conditioning device that provides conditioned air for a space to be air conditioned, which conditioned air provides the impression of fresh air and eliminates harmful components of the air to be conditioned.

This object is achieved by an air-conditioning device for a space to be air-conditioned. The air-conditioning device has a housing with an air supply and an air discharge. At least one flow path is formed between the air supply and the air discharge. In the flow path at least one heat exchanger, a blower, and a means for generating an excited gas are provided. The means for producing an excited gas is a piezoelectric transformer.

It is a further object of the present invention to provide a motor vehicle with an air-conditioning device that provides conditioned air for an interior space of the motor vehicle to be air conditioned, which conditioned air provides the impression of fresh air and eliminates harmful components of the air to be conditioned.

This object is achieved by a motor vehicle with an air-conditioning device for an interior space of a motor vehicle to be air-conditioned. According to a possible embodiment the air-conditioning device comprises a housing with an air supply and an air discharge. Between the air supply and the air discharge, a flow path for the air to be conditioned is formed. In the flow path, at least one heat exchanger and a blower are provided. According to the invention, a piezoelectric transformer for generating an excited gas is arranged in the flow path of the air-conditioning device. Further, a surface coating is provided in the flow path of the excited gas, which surface coating serves for the catalytic reduction of the ozone content of the excited gas and is arranged downstream of the means for generating the excited gas.

The means for generating the excited gas or gas mixture may be a plasma generator, an ionizer, or an ozone generator. According to a preferred embodiment, the means for generating the excited gas or gas mixture comprises a piezoelectric transformer.

A piezoelectric transformer (PT) is a type of resonance transformer based on piezoelectricity and, unlike the conventional magnetic transformers, is an electromechanical system. It serves to convert a supplied alternating electrical voltage of a certain frequency, which is determined by the mechanical dimensions of the transformer, into a higher or lower alternating voltage.

Piezoelectric transformers generate high electric fields via the piezoelectric effect. These fields are capable of ionizing gases and liquids by electrical excitation. On the secondary side of the PT, the alternating electric field generates a strong polarization, excitation, and ionization of atoms and molecules. This process generates a piezoelectrically ignited microplasma, Piezoelectric Direct Discharge Plasma (PDD). PDDs have properties that correspond to the typical dielectric barrier discharges (DBD). PDDs can be ignited in a wide pressure range of 0.01 mbar and 2000 mbar.

Parasitic discharge phenomena on the piezoelectric transformer are undesirable, but this effect can also be used selectively. With PDD, a plasma can be ignited directly. Similar to a silent electrical discharge, when the oscillating field strengths are sufficiently high, a cold discharge occurs. Due to the high field inhomogeneity and the frequency influence, the surrounding gas can be ionized even under atmospheric conditions without the absolute ignition voltage having to be below the Paschen curve for this purpose.

To produce PDD plasma, Rosen type piezoelectric transformers are particularly suitable since this type provides high power densities and very high transformation ratios. Transformation ratios of more than 1000 can be achieved in practice. Resonant frequencies between 10 kHz to 500 kHz are optimal for igniting PDD plasma. If the power driver is optimally adapted to the resonance and the impedance of the piezoelectric transformer, the conversion into the discharge process takes place with high efficiency in the overall system.

Ozone generators based on PDD and operated with air provide a mean ozone concentration with the highest efficiency of the hitherto known systems. The gas temperature in the plasma volume in PDD is typically at an ambient temperature of 300 K to 320 K. Electron densities of about 1014 and 1016 m⁻³ are achieved. Thus, PDD provides a typical “cold” non-equilibrium plasma. These properties of PDD open up a wide range of possible applications. PDD devices are used in medical research, for germ reduction, for odor reduction, and in microbiology. Typical industrial applications comprise surface activation to optimize wetting and adhesion properties of plastics, e.g., in printing, painting, and adhesive processes.

The air-conditioning device may also comprise a means for increasing the activity/reactivity disposed in the flow path in the housing of the air-conditioning device. The excited gas generating means is provided between the blower and the activity/reactivity increasing means. According to a further embodiment of the air-conditioning device, the blower is arranged in the flow direction of the air of the means for increasing the activity/reactivity. Another possibility is that, in the flow direction of the air, the blower is arranged downstream of the means for increasing the activity/reactivity.

According to another embodiment of the air-conditioning device according to the invention, a filter is provided within the housing, which filter is upstream of the heat exchanger in the flow direction of the air. The means for generating an excited gas is then upstream of the filter in the flow direction of the air or downstream of the filter in the flow direction of the air.

The means for increasing the activity/reactivity may be a catalyst structure, an activated carbon structure, or a reactive filter. In this case, the means for increasing the activity/reactivity has a catalytically active surface, by means of which heterogeneous oxidation molecules and microorganisms are degraded.

A distributor flap is arranged upstream of the air supply into the housing of the air-conditioning device, which distributor flap is selectively either in fluid communication with an ambient air or an internal air of the space to be air-conditioned. A filter for particles, dirt, or water may be arranged upstream of the flap.

A distributing system can be arranged downstream of the air discharge out of the housing of the air-conditioning device, so that the conditioned air can be distributed in the space to be conditioned in the desired manner.

Of particular advantage is the use of the air-conditioning device according to the invention for the air-conditioning of the interior of motor vehicles. By the appropriate positioning of a plasma generator, an ionizer or ozone generator via electrical gas discharge in the flow path of the air in the air-conditioning system for a motor vehicle, a reactive gas or gas mixture can be generated, which together with other components of the air-conditioning device provides for a supply of the passenger compartment of motor vehicles with fresh air free of odors and harmful molecules or microorganisms. One of the components is, for example, the filter element, which may already contain reactive constituents or catalytically active constituents, in order to reduce the ozone content of the air in the air-conditioning device. Likewise, the heat exchanger may have a surface coating, which catalytically degrades the ozone content of the air in the flow path. The degradation of harmful gas species (molecules, microorganisms) in the air flow in the housing of the air-conditioning device can be carried out by heterogeneous oxidation on the catalytically active surfaces. With all these measures, one obtains a supply of conditioned air in the interior of a motor vehicle, which conditioned air is substantially clean and provides the impression of fresh air.

The means for increasing the activity/reactivity is, in one embodiment, provided with activated carbon forming the activated carbon structure. The activated carbon may, for example, be impregnated, such as with transition metals, platinum group metals, and/or manganese oxide. Activated carbon or manganese oxide can also be applied, for example, as a layer material to any desired surface. In one embodiment, the catalyst structure of the means for increasing the activity/reactivity is formed on a cooler (condenser) and has a hydrophobic configuration in order to obtain a high active surface even under moist conditions (analogous to a PEM fuel cell). These surfaces may be formed, for example, on the heat exchanger, the blower/evaporator blower, and the inner walls of the air distribution channels or on the distributing system.

A conventional heat exchanger typically cools the air to be conditioned down to about 5° C., thereby drying the air to 5° C. dew point. This cold and dry state of the air is particularly suitable for, for example, generating high ozone concentrations with high efficiency by the means for generating an excited gas, favored by a low temperature and a low humidity. In general, the air cooled and dried after the cooling process by means of the cooler part (evaporator) of the heat exchanger is again heated to approximately 20° C., for example, by means of a suitable electrical structure. This heated structure can be catalytically particularly active. The heat exchanger comprises in its entirety the cooler (evaporator/condenser) and an (additional) heater (for example, heated structure) and has a very large surface, which is suitable for a surface coating in the context of the invention.

However, in the above surface coating, when applied to a surface of the evaporator, it is problematic that liquid condenses out there. The surface thus gets wet and the activity of the catalyst decreases or even ceases to exist. To avoid this, the catalyst coating should preferably be provided with a hydrophobic admixture, for example with Teflon and/or other hydrophobic materials. After the evaporation process by means of the evaporator, the surfaces are dry again. In addition, other surfaces can be catalytically equipped, for example, walls of the entire flow guidance (distributing system), or especially where high turbulence arises, so for example at the blower. The surface of the (additional) heater is heated during operation, whereby a catalyst applied there would cause high activity.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 is a schematic view of an embodiment of the air-conditioning device according to the invention;

FIG. 2 is a schematic view of another embodiment of the air-conditioning device according to the invention;

FIG. 3 is a schematic view of another embodiment of the air-conditioning device according to the invention;

FIG. 4 is a schematic view of another embodiment of the air-conditioning device according to the invention;

FIG. 5 is a schematic view of another embodiment of the air-conditioning device according to the invention;

FIG. 6 is a schematic view of another embodiment of the air-conditioning device according to the invention; and,

FIG. 7 is a schematic representation of the use of the air-conditioning device according to the invention in a motor vehicle.

### DETAILED DESCRIPTION

At the outset, it should be appreciated that like drawing numbers on different drawing views identify identical, or functionally similar, structural elements. It is to be understood that the claims are not limited to the disclosed aspects.

The illustrated embodiments are only examples of how the air-conditioning device according to the invention can be configured and is not to be understood as a final restriction. The proportions of the individual elements to one another in the figures do not always correspond to the actual size ratios, since some shapes are simplified and other shapes are shown enlarged in relation to other elements for better illustration.

Furthermore, it is understood that this disclosure is not limited to the particular methodology, materials, and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

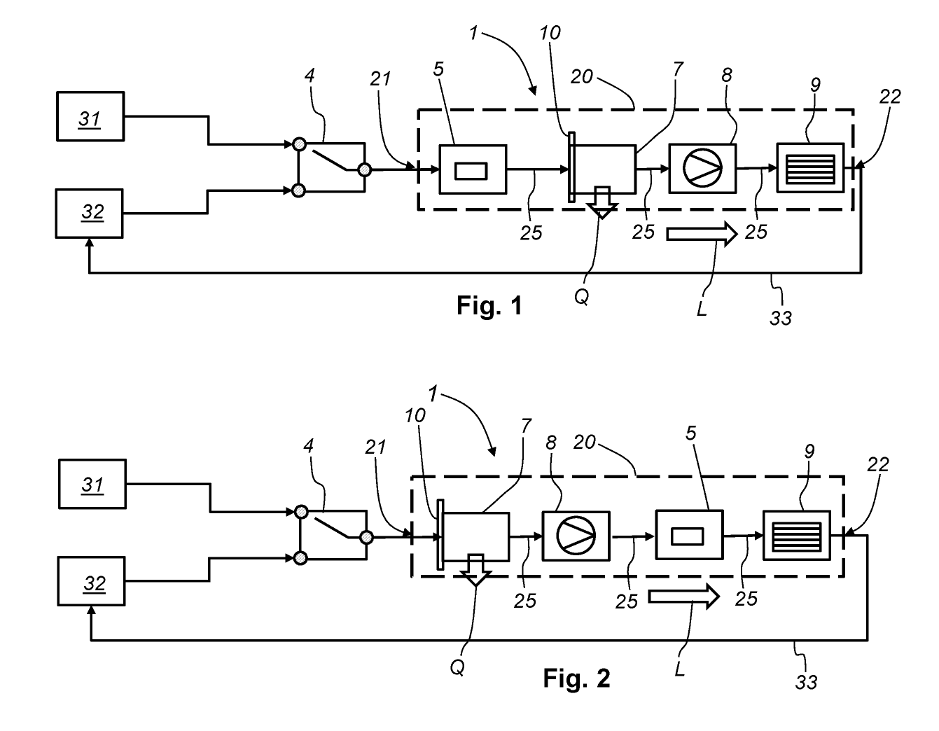

FIG. 1 schematically shows the structure of air-conditioning device 1, which is preferably used for air-conditioning of the passenger compartment or interior of motor vehicles. Air-conditioning device 1 comprises housing 20 (shown in dashed lines in FIGS. 1-6). Housing 20 has formed air supply 21 (for the air not conditioned) and air discharge 22 (conditioned and purified air). Between air supply 21 and air discharge 22, flow path 25 is defined. In the embodiment shown in FIG. 1, in flow path 25 in flow direction L in housing 20 downstream of air supply 21, means for generating an excited gas 5, heat exchanger 7, blower 8, and means for increasing the activity/reactivity 9 are provided. Via heat exchanger 7, heat Q is withdrawn from the air to be conditioned and discharged to the environment.

According to one embodiment, surface coating 10 is provided, for example, on heat exchanger 7. Surface coating 10 serves for catalytic degradation of the ozone content or the excited gas or plasma. The attachment of surface coating 10 on heat exchanger 7 has the advantage that there is a large surface area available to achieve the desired effect of the catalytic degradation. Other possibilities for providing surface coating 10 are provided by blower 8 and/or the evaporator blower. Here, the sufficient effect for the catalytic degradation is achieved by the high turbulence of the air to be conditioned. Another way of applying surface coating 10 is provided by the inner walls (not shown) of distribution system 11. This has the advantage that in distribution system 11, a large surface is available anyway, without the need for additional components for the application of the surface coating 10.

Distributor flap 4 is arranged upstream of air supply 21 of housing 20, so that selectively ambient air 31 or internal air 32 can be supplied to housing 20 for air-conditioning. From air discharge 22 of housing 20, the conditioned air is conducted into space 33 to be air-conditioned, which in the illustration shown in FIG. 1 is represented by an arrow to internal air 32. Means for generating an excited gas 5 is a piezoelectric transformer which functions as a plasma generator, an ionizer, or an ozone generator. The excited gas achieves a sterilization and a fresh effect of the air to be conditioned.

FIG. 2 shows a further embodiment of air-conditioning device 1 according to the invention. Here, another arrangement of the elements of air-conditioning device 1 causing the air-conditioning is illustrated within housing 20. Air supply 21 is followed by heat exchanger 7, which is provided with surface coating 10. Then, further provided in flow direction L in flow path 25 are blower 8, means for generating an excited gas 5, and means for increasing the activity/reactivity 9.

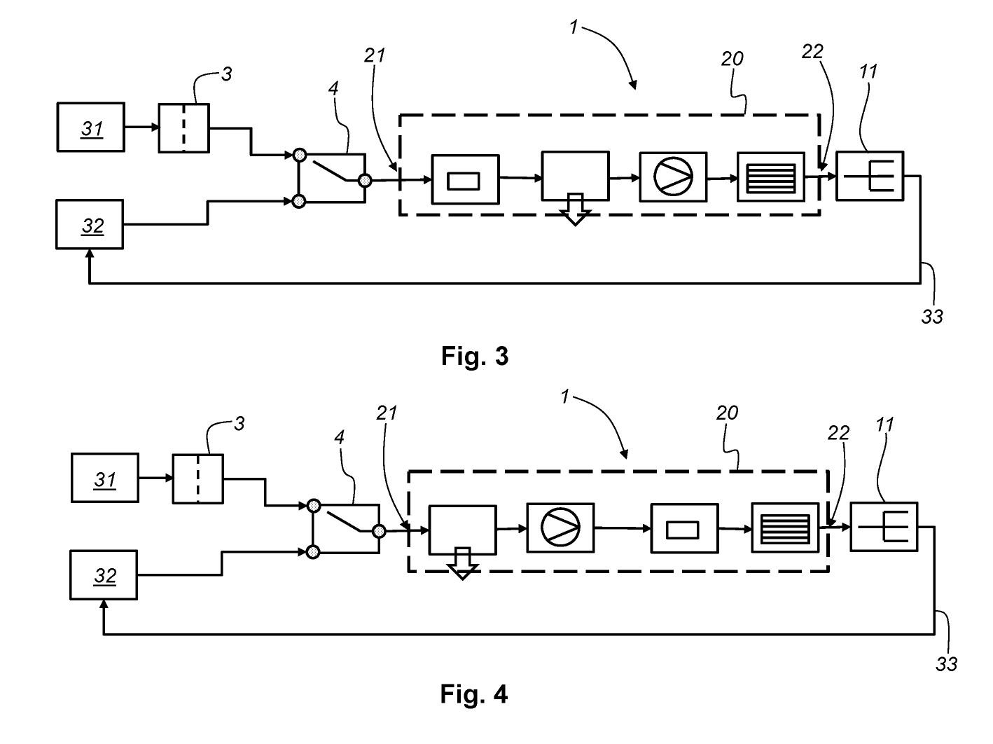

FIGS. 3 and 4 show embodiments of the invention wherein air discharge 22 is arranged upstream of distributing system 11. With the distribution system, the conditioned air can be distributed in a targeted manner in space 33 to be air-conditioned. The arrangement of the elements in housing 20 of the embodiment described in FIG. 3 corresponds to the arrangement as described in FIG. 1. Likewise, the arrangement of the elements in housing 20 of the embodiment described in FIG. 4 corresponds to the embodiment of the arrangement as described in FIG. 2. Likewise, distributor flap 4 and filter 3 of ambient air 31, for particles, dirt, or water, are arranged upstream of air supply 21 in the embodiments shown in FIGS. 3 and 4.

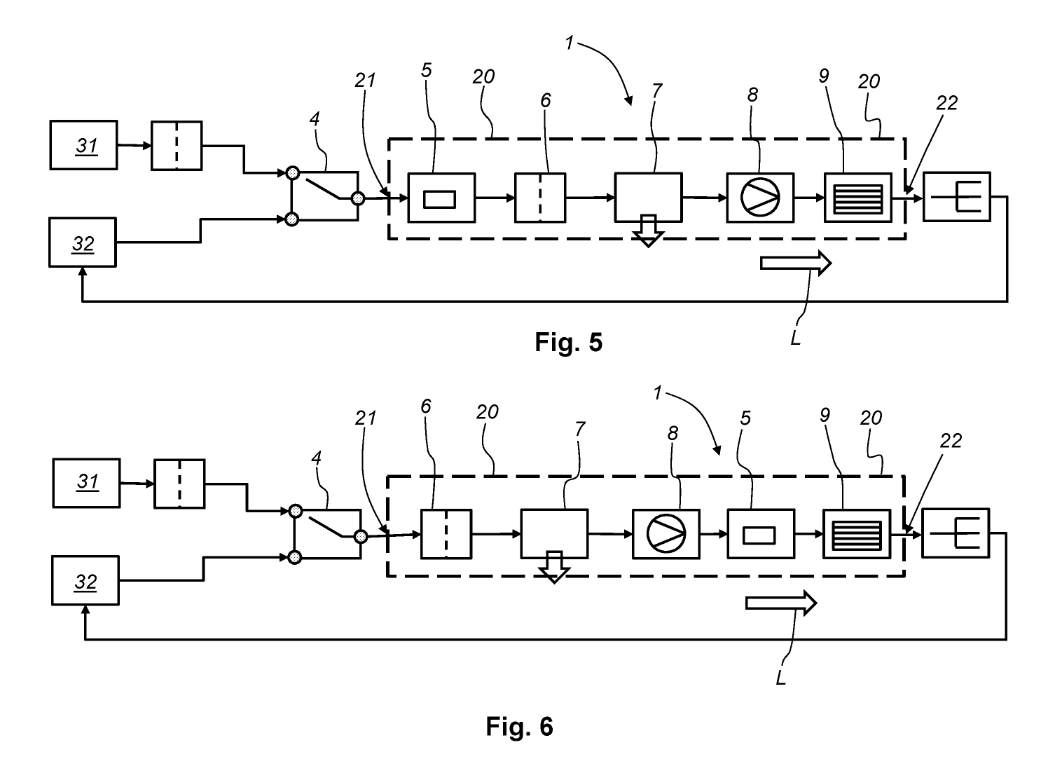

FIG. 5 shows a further embodiment of air-conditioning device 1. In housing 20, in flow direction L, means for generating an excited gas 5, filter 6, heat exchanger 7 (possibly with a surface coating, not shown here), blower 8, and means for increasing the activity/reactivity 9 are arranged between air supply 21 and air discharge 22. Filter 6 may already contain reactive constituents or catalytically active constituents in order to reduce the ozone content of the air to be conditioned.

FIG. 6 shows a modification of the embodiment shown in FIG. 5. In housing 20, in flow direction L, filter 6, heat exchanger 7 (possibly with a surface coating, not shown here), blower 8, means for generating an excited gas 5, and means for increasing the activity/reactivity 9 are arranged between air supply 21 and air discharge 22.

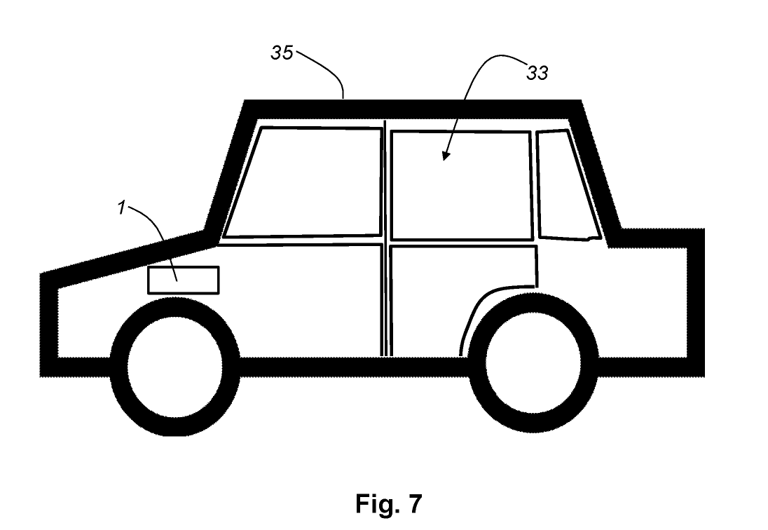

FIG. 7 shows a schematic representation of the use of air-conditioning device 1 according to the invention in motor vehicle 35 for air-conditioning. Space 33 to be air-conditioned is the interior or the passenger compartment of motor vehicle 35.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims. All of the above-described embodiments of the air-conditioning device can be used both individually and in any combination.

### LIST OF REFERENCE NUMERALS

1 Air-conditioning device
3 Filter
4 Distributor flap
5 Means for generating an excited gas
6 Filter
7 Heat exchanger
8 Blower
9 Means for increasing the activity/reactivity
10 Surface coating
11 Distributing system
20 Housing
21 Air supply
22 Air discharge
25 Flow path
31 Ambient air
32 Internal air
33 Space
35 Motor vehicle
L Flow direction
Q Heat

What is claimed is:

1. An air-conditioning device for a space to be air-conditioned, comprising:
a housing including an air supply, an air discharge, and at least one flow path formed between the air supply and the air discharge; and,
at least one heat exchanger, a blower, and a means for generating an excited gas arranged in the at least one flow path.

2. The air-conditioning device as recited in claim 1, wherein the means for generating the excited gas comprises a piezoelectric transformer.

3. The air-conditioning device as recited in claim 1, wherein the means for generating the excited gas comprises a plasma generator, an ionizer, or an ozone generator.

4. The air-conditioning device as recited in claim 1, wherein a surface coating is arranged downstream of the means for generating the excited gas in the at least one flow path for the catalytic reduction of the ozone content of the excited gas.

5. The air-conditioning device as recited in claim 1, wherein a means for increasing the activity/reactivity is arranged in the at least one flow path in the housing.

6. The air-conditioning device as recited in claim 5, wherein the means for generating the excited gas is arranged in the at least one flow path between the blower and the means for increasing the activity/reactivity.

7. The air-conditioning device as recited in claim 6, wherein the blower is arranged upstream of the means for increasing the activity/reactivity in the at least one flow path, or the blower is arranged downstream of the means for increasing the activity/reactivity in the at least one flow path.

8. The air-conditioning device as recited in claim 1, further comprising a filter arranged in the housing and upstream of the at least one heat exchanger in the at least one flow path.

9. The air-conditioning device as recited in claim 8, wherein the means for generating the excited gas is arranged upstream of the filter in the at least one flow path or is arranged downstream of the filter in the at least one flow path.

10. The air-conditioning device as recited in claim 5, wherein a catalyst structure, an activated carbon structure, or a reactive filter is arranged upstream of the means for increasing the activity/reactivity in the at least one flow path.

11. The air-conditioning device as recited in claim 10, wherein the means for increasing the activity/reactivity has a catalytically active surface by means of which molecules are degradable through heterogeneous oxidation.

12. The air-conditioning device as recited in claim 10, wherein the means for increasing the activity/reactivity has a catalytically active surface by means of which microorganisms are inhibited in growth or killed through heterogeneous oxidation.

13. The air-conditioning device as recited in claim 1, further comprising a distributor flap arranged upstream of the air supply into the housing, the distributor flap is selectively in fluid communication with an ambient air or an internal air of the space.

14. The air-conditioning device as recited in claim 13, further comprising a filter arranged upstream of the distributor flap to filter the ambient air.

15. The air-conditioning device as recited in claim 1, further comprising a distributing system arranged downstream of the air discharge.

16. A motor vehicle, comprising:
an air-conditioning device for a space of the motor vehicle to be air-conditioned, the air-conditioning device comprising:
a housing including an air supply, an air discharge, and at least one flow path formed between the air supply and the air discharge;
at least one heat exchanger, a blower, and a piezoelectric transformer for generating an excited gas arranged in the at least one flow path; and,
a surface coating arranged on the at least one heat exchanger and downstream of the piezoelectric transformer in the at least one flow path.

17. The motor vehicle as recited in claim 16, further comprising a means for increasing the activity/reactivity arranged in the at least one flow path in the housing.

18. The motor vehicle as recited in claim 17, wherein the blower is arranged upstream of the means for increasing the activity/reactivity in the at least one flow path.

19. The motor vehicle as recited in claim 16, wherein:
a filter is arranged in the housing and upstream of the heat exchanger in the at least one flow path; and,
the piezoelectric transformer is arranged upstream of the filter in the at least one flow path or is arranged downstream of the filter in the at least one flow path.

20. An air-conditioning device for a space to be air-conditioned, comprising:
a housing including an air supply, an air discharge, and at least one flow path formed between the air supply and the air discharge;
at least one heat exchanger, a blower, and a piezoelectric transformer for generating an excited gas arranged in the at least one flow path;
a surface coating arranged downstream of the piezoelectric transformer in the at least one flow path; and,
a means for increasing the activity/reactivity arranged in the at least one flow path in the housing;
wherein the piezoelectric transformer is arranged between the blower and the means for increasing the activity/reactivity.
